(19) United States
(12) Patent Application Publication
(10) Pub. No.: US 2022/0193333 A1
(43) Pub. Date: Jun. 23, 2022

(54) BATTERY ANTENNA ARRANGEMENT FOR AN ON BODY MEDICAL DEVICE

(71) Applicant: Insulet Corporation, Acton, MA (US)

(72) Inventors: Nicholas CONTE, Harvard, MA (US); Kepei SUN, Andover, MA (US)

(21) Appl. No.: 17/553,713

(22) Filed: Dec. 16, 2021

Related U.S. Application Data
(60) Provisional application No. 63/127,323, filed on Dec. 18, 2020.

(51) Int. Cl. A61M 5/142; H01M 10/04; H01Q 1/22; A61M 5/145

(52) U.S. Cl. CPC A61M 5/14248; H01M 10/0427; A61M 2205/8206; A61M 5/145; H01Q 1/22

(57) ABSTRACT

One or more button cell batteries may be used in an on-body medical device like a drug delivery device to act as an antenna for wireless communications. Since the one or more batteries are already present in the on-body medical device, no additional real estate on the printed circuit board is required for the antenna. In some exemplary embodiments, a single button cell battery is used as an antenna, and in other embodiments, multiple button cell batteries are used as the antenna. For example, a single button cell battery may be used as part of a monopole antenna. Multiple button cell batteries may be used as part of a dipole antenna.

### RELATED APPLICATIONS

This application claims the benefit of U.S. Provisional Patent Application No. 63/127,323, filed Dec. 18, 2020, the contents of which are incorporated herein by reference in their entirety.

### BACKGROUND

Some conventional medical devices that are worn on body have a wireless communication capability. For example, certain glucose monitors have a Bluetooth® communication capability. In order to provide such a wireless communication capability, these on-body medical devices include an antenna. A typical approach for such conventional on-body medical devices has been to provide an antenna on a printed circuit board inside the housing of the on-body medical device. For example, a strip antenna may be formed on the printed circuit board, or an antenna may be surface mounted on the printed circuit board.

There are a few drawbacks to these conventional approaches of providing the antenna on the printed circuit board. First, the antenna may occupy a large area on the printed circuit board. Given that a printed circuit board for such a medical device typically is small and that space on the printed circuit board is a valuable resource, using the area on the printed circuit board for the antenna wastes the valuable resource. In some instances, the size of the printed circuit board may need to be increased to accommodate the antenna. Second, such strip antennas and surface mounted component antennas on a printed circuit board are known to be of low efficiency when the printed circuit board is attached in very close proximity to the body of a user. This low efficiency may result in intermittent loss of communication capability and an unsatisfying user experience. Third, since the antennas are either formed directly on the printed circuit board or surface mounted on the printed circuit board, the other components on the printed circuit board must be arranged so that they do not block or obstruct transmission/reception of communications with the antenna.

### SUMMARY

In accordance with an inventive aspect, a drug delivery device includes one or more button cell batteries for powering at least a portion of the drug delivery device. Each button cell battery of the one or more button cell batteries is cylindrical and has a longitudinal axis. The drug delivery device also includes a wireless communication transceiver for transmitting and receiving wireless communications. In addition, the drug delivery device includes an electrical connection between the wireless communication transceiver and the one or more button cell batteries so that the one or more button cell batteries act as an antenna that transmits wireless communications from the wireless communication transceiver and receives wireless communications destined for the wireless communication transceiver. The drug delivery device also includes a housing that is configured to be secured to a body of a user so that the longitudinal axis of the at least one button cell battery is substantially perpendicular to a surface of the body of the user where the housing is secured.

In some embodiments, there may be a single button cell battery and in other embodiments there may be multiple button cell batteries. The drug delivery device may be configured to emit surface waves from the one or more button cell batteries for travelling along the surface of the body of the user. The drug delivery device may include a printed circuit board on which the one or more button cell batteries are positioned and where a ground plane is formed. The wireless communication transceiver may be, for example, a Bluetooth transceiver, a Bluetooth Low Energy transceiver, a Body Area Network (BAN) transceiver, or a WiFi transceiver. The drug delivery device may further include at least one battery holder for holding the one or more button cell batteries. The electrical connection between the wireless communication transceiver, and the one or more button cell batteries may be connected to the at least one battery holder, which is in electrical contact with the one or more button cell batteries.

In accordance with an inventive aspect, a drug pump includes one or more button cell batteries for powering at least a portion of the drug pump. The drug pump may be used to pump insulin, or glucagon, or another type of drug into the body of a user. Each of the one or more button cell batteries is cylindrical and has a longitudinal axis. The insulin pump also includes a wireless communication transceiver for transmitting and receiving wireless communications. The insulin pump additionally includes an electrical connection between the wireless communication transceiver, and the one or more button cell batteries so that the one or more button cell batteries act as an antenna that transmit(s) wireless communications from the wireless communication transceiver and receive(s) wireless communications destined for the wireless communication transceiver. The drug pump further includes a housing that is configured to be secured to a body of a user so that the longitudinal axes of the one or more button cell batteries are substantially perpendicular to a surface of the body of the user to which the housing is secured.

In some embodiments there is a single button cell battery and in other embodiments there are multiple button cell batteries. The drug pump may be configured to emit surface waves from the one or more button cell batteries for travelling along the surface of the body of the user. The drug pump may include a printed circuit board on which the one or more button cell batteries are positioned and where a ground plane is formed. The transceiver may be a Bluetooth transceiver, a Bluetooth Low Energy transceiver, a Body Area Network (BAN) transceiver, or a WiFi transceiver. The insulin pump may include at least one battery holder for holding the one or more button cell batteries. The electrical connection between the wireless communication transceiver and the one or more button cell batteries may be connected to the at least one battery holder, which are in electrical contact with the one or more button cell batteries.

In accordance with another inventive aspect, a method is practiced where at least one button cell battery is positioned on a printed circuit board in a drug delivery device. The at least one button cell battery is electrically connected to the printed circuit board to provide power for the drug delivery device. A wireless communication transceiver is electrically and mechanically connected to the printed circuit board. An electrical feed is connected between the at least one button cell battery and the wireless communication transceiver to create an antenna for transmitting wireless communications from the wireless communication transceiver and receiving wireless communications for the wireless communication transceiver.

The method may further include electrically and mechanically connecting to the printed circuit board at least one battery holder for the at least one button cell battery. The at least one button cell battery may be held by the at least one battery holder so as to be electrically connected with the at least one battery holder, and the electrical feed may be connected to the at least one battery holder so as to be electrically connected with the at least one button cell battery. The wireless communication transceiver may be a Bluetooth® transceiver, a Bluetooth® Low Energy (BLE) transceiver, a Body Area Network (BAN) transceiver, or a WiFi transceiver.

### BRIEF DESCRIPTION OF THE DRAWINGS

FIG. 1A depicts a block diagram of a drug delivery device of a drug delivery device and a user for an exemplary embodiment.

FIG. 1B depicts a more detailed block diagram of the printed circuit board of FIG. 1A.

FIG. 1C depicts a partially exploded side view of a drug delivery device of an exemplary embodiment.

FIG. 2 depicts a side view of layers of a printed circuit board for a drug delivery device of an exemplary embodiment.

FIG. 3 depicts an arrangement where a single button cell battery is used as an antenna for a drug delivery device in an exemplary embodiment.

FIG. 4 depicts an illustrative gain plot for a monopole antenna using a single button cell battery in a drug delivery device of an exemplary embodiment.

FIG. 5 depicts a flowchart of illustrative steps that may be performed to form an antenna in an exemplary embodiment.

FIG. 6 depicts a block diagram of an illustrative drug delivery system that includes an insulin pump as a drug delivery device for an exemplary embodiment.

FIG. 7 depicts an exemplary drug delivery system for an exemplary embodiment.

### DETAILED DESCRIPTION

Exemplary embodiments may use one or more batteries in an on-body medical device to act as an antenna for wireless communication. Since the one or more batteries are already present on a printed circuit board of the on-body medical device to provide power, no additional space on the printed circuit board is required for the antenna. Use of the batteries to form the antenna may also enable the printed circuit board for the on-body medical device to be smaller and thus enable the on-body medical device to be smaller. In some exemplary embodiments, a single button cell battery is used as the antenna, and in other embodiments, multiple button cell batteries are used as the antenna. For example, a single button cell battery may be used as part of a monopole antenna. Multiple button cell batteries may be used as part of a dipole antenna. In the case of a single button cell battery used as part of a monopole antenna, a single button cell battery or multiple button cell batteries may be used to power the on-body medical device, with one being used concurrently as a monopole antenna. In alternative embodiments, the batteries need but be button cell batteries but may be other varieties of batteries may be used. More generally, batteries that are flat with thin structures like a disk or coin may be suitable.

In addition, the antennas of the exemplary embodiments may be configured to not suffer from the inefficiencies of conventional surface mounted antennas that are mounted on printed circuit boards or trace antennas formed on printed circuit boards. Some of the inefficiencies may result from the conventional trace antennas or surface mounted antennas being oriented parallel to the body of the user and as a result, a great deal of the transmitted energy from such conventional antennas may be absorbed by the body of the user. The human body is a lossy medium for electromagnetic waves, and the resulting loss due to absorption by the human body may greatly influence antenna performance. The antennas of the exemplary embodiments may be configured to be oriented substantially perpendicular to the body surface of the user so that less energy of the transmitted signals is absorbed by the human body. Antennas placed perpendicular to the human body suffer less absorption by the human body. Some of the inefficiencies of conventional surface mounted antennas and trace antennas formed on the printed circuit board also have to do with the minimal separation of the antennas from the surface of the body of the user. This may be addressed by placing greater separation between the button cell batteries of the antennas of the exemplary embodiments and the body surface of the user by, for example, design of the housing of the on-body medical device.

The exemplary embodiments may provide antennas that are well suited for wireless communications between multiple on-body devices as well as between an on-body device or devices and an off-body device or devices. The antennas of the exemplary embodiments may transmit surface waves that travel along an outer body surface of the user that are well suited for quality communications with other on-body devices. In addition, the antennas of the exemplary embodiments may transmit electromagnetic waves with sufficient energy in an off-body direction to facilitate quality communications with off-body devices.

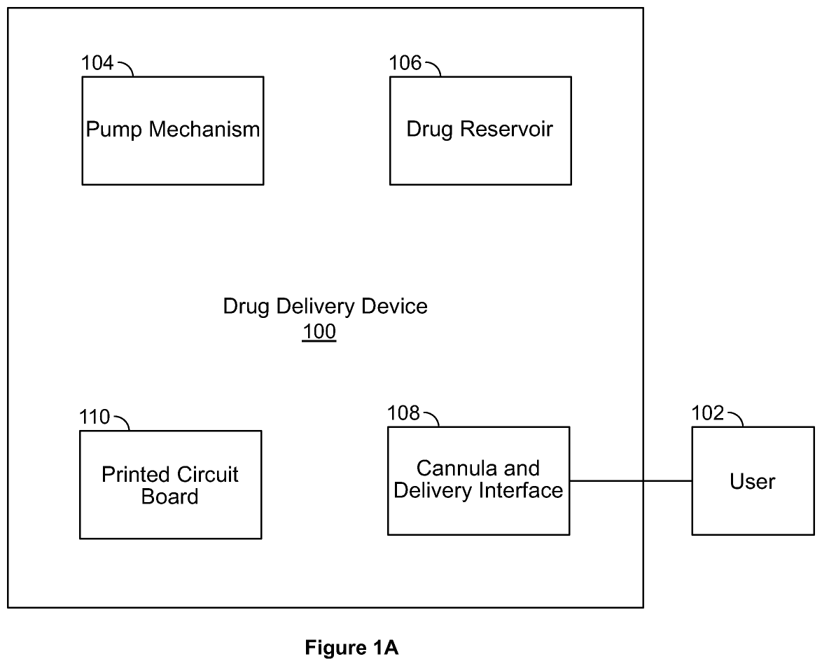

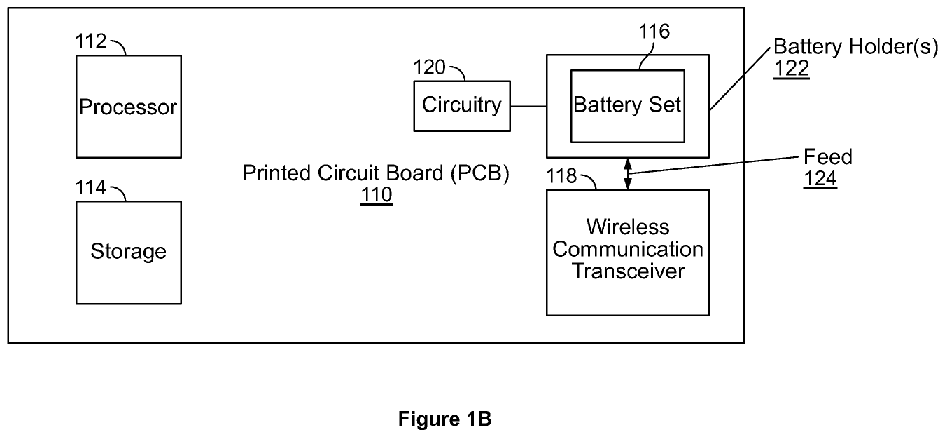

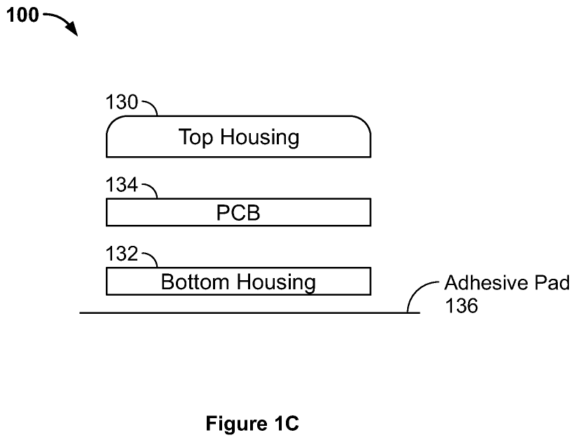

FIG. 1A depicts a block diagram of an illustrative drug delivery device 100 of an exemplary embodiment. In one exemplary embodiment, the drug delivery device 100 delivers insulin to a user 102. The drug delivery device 100 is worn on body by the user 102. The drug delivery device 100 may be secured to the user 102 using securing mechanisms like straps, adhesives, a body conforming housing or the like. A pump 104 may be provided for pumping drug stored in a drug reservoir 106 to the user 102. The pump 104 may be, for example, a reciprocating pump or a positive pressure pump. A cannula/needle and delivery interface 108 may be provided. The cannula/needle may pierce the skin of the user 102 and provide a pathway along with fluid conduits (such as tubing) for delivery of the drug to user 102. The drug delivery device 100 delivers the drug to the user 102 under programmatic control. The drug delivery device 100 includes at least one printed circuit board (PCB) 110 on which various electronic components may be positioned.

FIG. 1B shows a block diagram that depicts more detail of the PCB 110. The PCB 110 has a processor 112 positioned on it. The processor 112 controls operation of the drug delivery device. For example, the processor 112 may control how much drug is delivered to the user 102 and when the drug is delivered. The processor 112 may take many different forms including as a central processing unit (CPU), a graphics processing unit (GPU), an applications specific integrated circuit (ASIC), a field programmable gate array (FPGA), a special purpose controller chip or a system on a chip (SoC). The processor 112 may execute programming instructions stored in storage 114. The storage 114 may include one or more types of storage including but not limited to random access memory (RAM), flash memory, read only memory (ROM), computer-readable memory storage and the like. The storage 114 may also hold data and other useful information for operation of the drug delivery device 100.

The PCB 110 may include a battery set 116 containing one or more batteries. The one or more batteries 116 may include button cell batteries. The batteries in the battery set 116 may be silver oxide batteries, alkaline batteries, zinc air batteries, lithium batteries of the like. The batteries in the battery set 116 may be cylindrical in shape as is typical off button cell batteries. The batteries in the battery set 116 may be of any of a number of diameters, such as found in commercially available button cell batteries. The batteries of the battery set 116 may be held by one or more battery holder(s) 122. The battery holder(s) 122 may be in electrical contact with the anodes and cathodes of the batteries of the battery set 116. Moreover, the battery holder(s) may be mechanically connected to the PCB 110 and may be electrically connected to the PCB 110.

In the exemplary embodiments, the battery set 116 provides power for components of the drug delivery device 100. In addition, the battery set 116 is used as a wireless antenna for transmitting and receiving wireless communications from other devices positioned on-body and off-body as will described in more detail below. A wireless communication transceiver 118 is provided to both transmit and receive wireless communications. The wireless communication transceiver 118 may transmit and receive communications in wireless formats, such as Bluetooth® Bluetooth®, Low Energy (BLE), WiFi or IEEE 802.15.6 Wireless Body Area Network (WBAN). An electrical feed 124 electrically connects the wireless communication transceiver 118 with the battery set 116, where the battery set 116 acts as a wireless antenna that transmits wireless communications from the wireless communication transceiver 118 and receives wireless communications that are forwarded to the wireless communication transceiver 118. The electrical feed 124 may be electrically connected to the battery holder(s) 122 in some embodiments and electrically connected to the battery set 116 in other embodiments. Electrical circuitry 120, such as a capacitor, may be provided to tune the impedance, provide filtering and the like. The electrical circuitry 120 may also include other electrical components.

FIG. 1C shows a side partially exploded view of the drug delivery device 100. The drug delivery device 100 may have a protective housing formed by a top housing 130 and a bottom housing 132. The top housing 130 and the bottom housing 132 may be secured together via a snap fit feature, via adhesive, via fasteners, such as screws or the like. The PCB 134 is positioned inside the interior space formed between the top housing 130 and the bottom housing 132 when the two housing components 130 and 132 are secured together. Features may be provided on the interior of the top housing 130 and the bottom housing 132 to support the PCB 134 and to hold the PCB 134 in a fixed orientation. Preferably the PCB 134 is oriented parallel to the skin surface of the user with the longitudinal axis of the batteries in the battery set 116 being oriented perpendicular to the PCB 134 and the skin surface of the user. The top housing 130 and the bottom housing 134 may be formed of materials, such as polycarbonate, plastic or the like. An adhesive pad 136 may be secured to the exterior surface of the bottom housing 132. The adhesive pad 136 has a substrate to which an adhesive is applied. The adhesive is used to secure the drug delivery device 100 to the skin surface of the user 102. The adhesive pad 136 also may have an adhesive applied to the side that faces the exterior surface of the bottom housing 132 to secure the adhesive pad 136 to the bottom housing 132. Alternatively, the substrate may be heat welded to the exterior surface of the bottom housing 132 or integrally formed as part of the bottom housing 132.

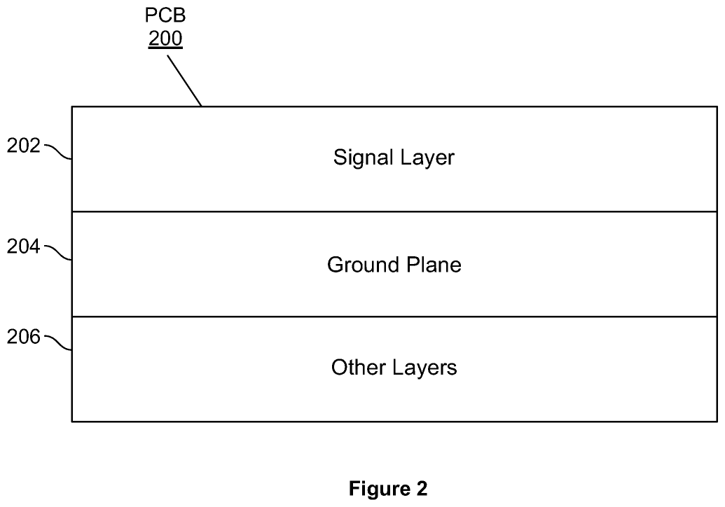

The ground plane for the antenna may be formed in the PCB 200 as shown in FIG. 2. The PCB 200 may be formed of multiple layers. In the example depicted in FIG. 2, the top layer of the PCB 200 is a signal layer 202 on which signaling tracks are formed on top of a dielectric. The next layer is a ground plane 204. The ground plane 204 may include a large metallized surface (such as a copper surface) tied to ground. Other layers 206 may also be present in the PCB 200. It is desirable for the antenna to have a large ground plane so as to improve the signal integrity of the antenna. The example depicted in FIG. 2 is intended to be illustrative and not limiting. Other PCB configurations with different layers and layer orders may be used.

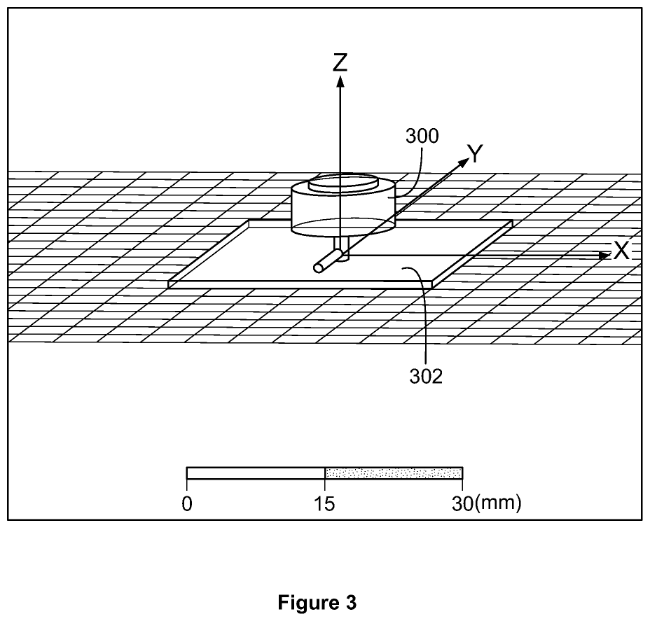

FIG. 3 shows a button cell battery 300 connected to the ground plane 302 in an antenna arrangement. The button cell battery has its flat surface positioned parallel to the surface of the PCB (i.e., the X-Y plane) and its longitudinal axis is positioned (along the Z axis) orthogonal to the skin surface of the PCB and the surface of the skin of the user. The ground plane 302 may be connected to the center of the surface of the button cell battery 300 bottom face that is parallel to the ground plane 302. The antenna has the battery acting as a radiating patch on one side of a dielectric in the PCB and the ground plane on the other side. With this arrangement, the antenna acts as a monopole antenna. FIG. 3 also depicts three axes, X, Y and Z. When the antenna is positioned on the skin surface of the user 102, the Z axis extends away from the skin surface of the user. The Y axis extends along a longitudinal direction of the user along the skin surface of the user from head to toe, and the X axis extends horizontally from one side or the other, such as across the skin surface of the user from their right side to their left side.

The antenna seeks to provide sufficient energy in transmissions along the Z axis so as to facilitate off-body communication and also seeks to provide sufficient energy in transmissions along the Y axis to facilitate transmissions along the skin surface of the user for on-body communications. The transmissions along the Y axis are configured to be surface waves. Surface waves tend to be entrained along a surface where there is a boundary condition formed between two mediums having different dielectric constants (i.e., different degrees of electrical permeability). The permeability of the air is much higher than the permeability of the human body. As a result, electrical signals travel faster in the air than in the human body. The net effect is the bottom portion of a propagating waveform tends to bend toward the skin surface of the user at the boundary between the air and the skin surface. The bending causes the waveform to be entrained along the surface of the skin of the user. This is desirable because the surface waves reach on-body devices better than wireless signals cast through the air or through the body of the user.

As was discussed above, conventional trace antennas formed on the PCB lack sufficient separation relative to the skin surface of the user. Moreover, the conventional trace antennas tend to direct a large amount of transmitted energy into the body of the user. The antennas described herein, by contrast, have greater separation relative to the skin surface of the user (e.g., from 2 mm up to 60 mm) because the battery set is positioned further form the PCB top surface. In addition, the directivity of the antenna has less energy transmitted toward the skin surface of the user because the antenna is oriented perpendicular to the skin surface of the user (see FIG. 3) and has a monopole distribution pattern.

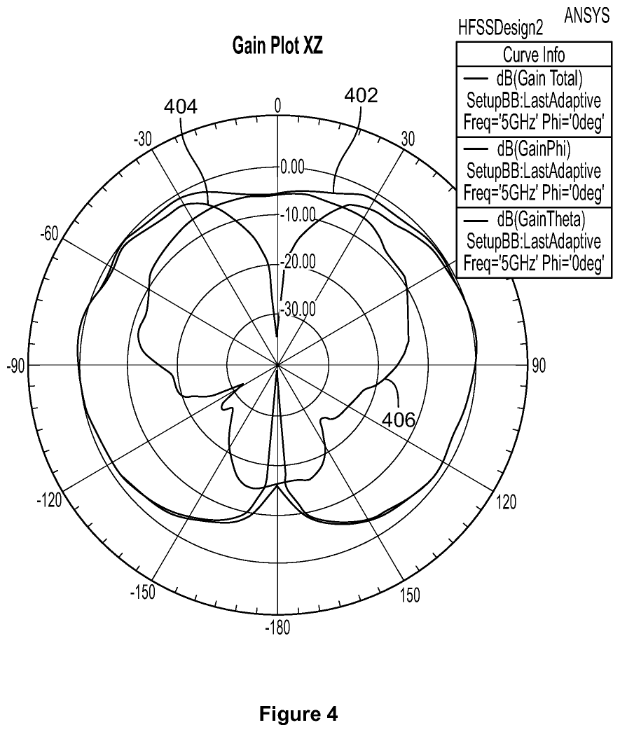

The single button cell arrangement of FIG. 3 acts a like a monopole circular patch antenna. FIG. 4 depicts a two-dimensional plot of the radiation pattern 400 for this antenna. The plot 400 shows a two-dimensional distribution of transmission energy expressed in decibels by angle of radiation relative to the antenna expressed in polar coordinates. Specifically, curve 402 is the total antenna gain pattern expressed in dbi, curve 404 is the antenna gain pattern for vertical to body polarization that is orthogonal to the planar surface of the PCB and the skin surface of the user, and curve 406 is the antenna gain pattern for the parallel to the planar surface of the PCB and the skin surface of the user. The plots show greater energy and more uniformly distributed energy in the direction parallel to the skin of the user and less energy and less uniform distribution in the direction orthogonal to the skin of the user. This distribution is the desired distribution discussed above.

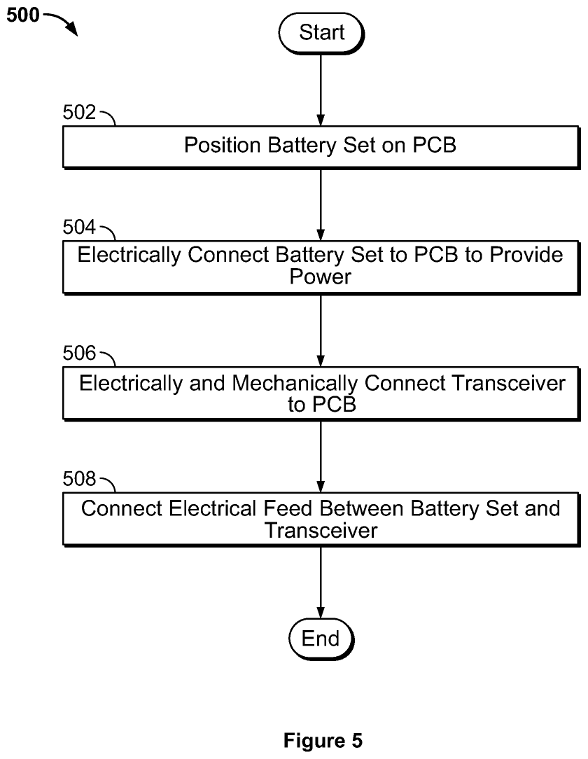

FIG. 5 depicts a flowchart 500 of the steps that may be performed in creating the antenna for an exemplary embodiment. The battery set 116 is positioned on the PCB (502). The battery set 116 may be held by one or more battery holders 122 that are electrically and mechanically connected to the PCB 110. The battery set 116 is electrically connected to the PCB 110 and provides power for the drug delivery device (504). The wireless communication transceiver 118 is electrically and mechanically connected to the PCB 110 (506). The wireless communication transceiver 118 may be an integrated circuit that is connected to the PCB 110 via pins or other connection approaches. An electrical feed 124 electrically connects the wireless communication transceiver 118 to the battery set 116 (508). As was mentioned above, the electrical feed 124 may be directly connected to the battery set 116 or instead to the battery holder(s) 122.

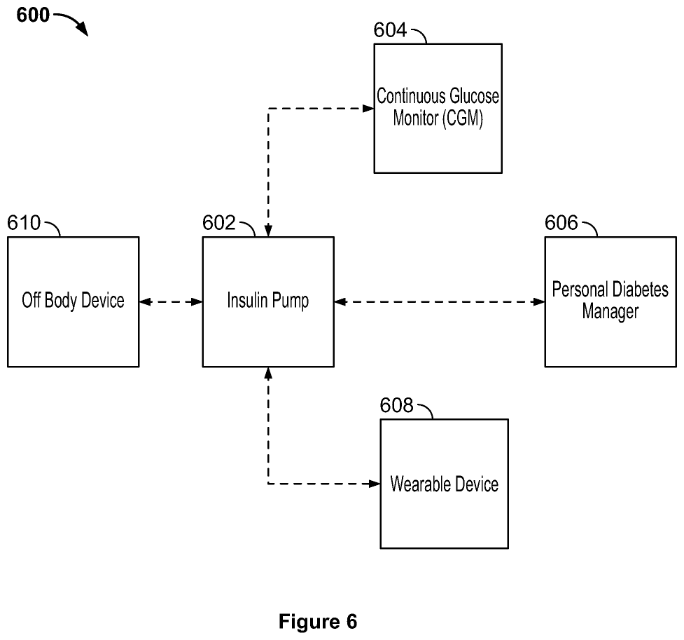

In an exemplary embodiment, the drug delivery device is an insulin pump. FIG. 6 shows an example of a drug delivery system 600 with such an insulin pump 602. The drug delivery system 600 includes different devices with which the insulin pump 602 may wirelessly communicate. These devices include an analyte monitor such as a continuous glucose monitor (CGM) 604 that provides blood glucose level readings on an ongoing basis. These readings may be sent to the insulin pump 602 wirelessly and used by a control algorithm of the insulin pump 602 to determine when and how much insulin is delivered to the user 102. The insulin pump 602 may also communicate with a remote device such as a smartphone or a personal diabetes manager (PDM) 606. The PDM 606 may be realized as a dedicated wireless device or as an application or other software running on a portable computing device, like a smartphone or tablet. The PDM 606 may serve as an interface with the user 102. The PDM 606 may provide the user 102 with information such as current analyte or blood glucose level as well as analyte (e.g., blood glucose) and drug (e.g., insulin) delivery history information and/or other informative content. The PDM 606 may also enable the user to control the insulin pump 602. The user 102 may modify certain settings by wirelessly communicating with the insulin pump 602 from the PDM 606. The insulin pump 602 may also wirelessly communicate with a wearable device 608, like a smart watch. The wearable device 608, for example, may receive and display information from the insulin pump. Moreover, the wearable device may be able to wirelessly issue commands for certain functionality to the insulin pump 602. The insulin pump 602 may also communicate with an off-body device 610, like an external device that understands a wireless protocol like those itemized above. All such wireless communications may be realized through the antenna formed with the battery set.

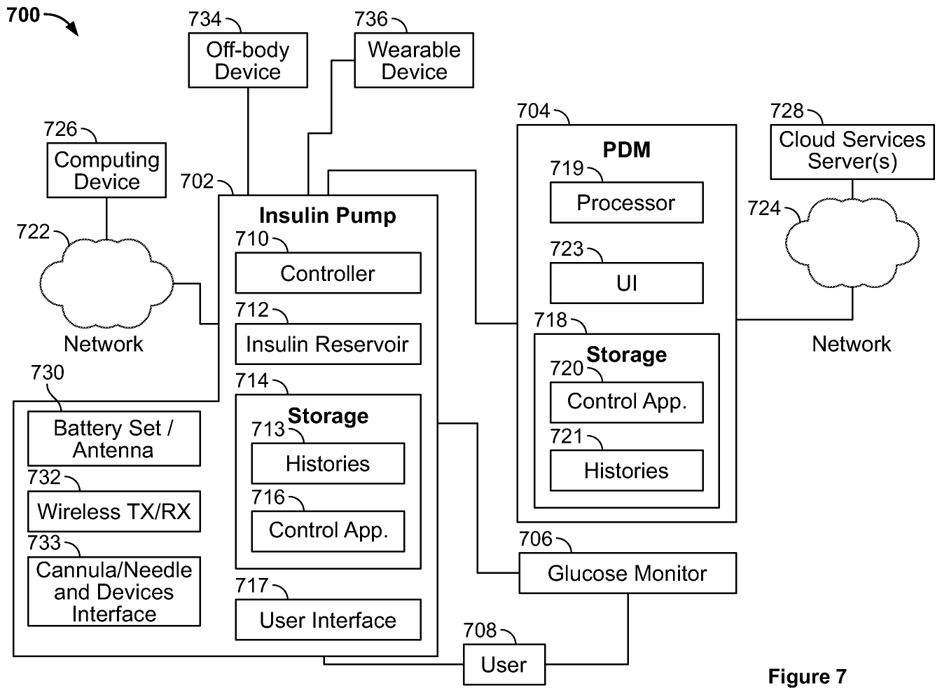

FIG. 6 only shows communication paths among components. It is helpful to see more detail regarding key components and to more fully discuss their functionality in order to appreciate why the antenna for wireless communications is helpful. To that end, FIG. 7 depicts additional detail for certain key components of an illustrative drug delivery system 700 in an exemplary embodiment. The drug delivery system 700 includes an insulin pump 702. As was mentioned above, the insulin pump 702 may be a wearable device that is worn on the body of the user 708. The insulin pump 702 may be directly coupled to a user (e.g., directly attached to a body part and/or skin of the user 708 via an adhesive or the like). In an example, a surface of the insulin pump 702 may include an adhesive to facilitate attachment to the user 708.

The insulin pump 702 may include a controller 710. The controller 710 may be implemented in hardware, such as processor 112 of FIG. 1B, software, or any combination thereof. The controller 710 may, for example, be a microprocessor, a logic circuit, a field programmable gate array (FPGA), an application specific integrated circuit (ASIC) or a microcontroller coupled to a memory. The controller 710 may maintain a date and time as well as other functions (e.g., calculations or the like). The controller 710 may be operable to execute a control application 716 stored in the storage 714 (see 114 in FIG. 1B) that enables the controller 710 to direct operation of the insulin pump 702. The storage 714 may hold histories 713 for a user, such as a history of automated insulin deliveries, a history of bolus insulin deliveries, meal event history, exercise event history and the like. In addition, the controller 710 may be operable to receive data or information. The storage 714 may include both primary memory and secondary memory. The storage may include random access memory (RAM), read only memory (ROM), optical storage, magnetic storage, removable storage media, solid state storage or the like.

The insulin pump 702 may include an insulin reservoir 712 (see drug reservoir 106 in FIG. 1A) for storing insulin for delivery to the user 708 as warranted. A fluid path to the user 708 may be provided, and the insulin pump 702 may expel the insulin from the insulin reservoir 712 to deliver the insulin to the user 708 via the fluid path. The fluid path may, for example, include the cannula/needle and delivery interface 733 (see 108 in FIG. 1A) and tubing coupling the drug pump 702 to the user 708 (e.g., tubing coupling a cannula to the insulin reservoir 712).

There may be one or more communications links with one or more devices physically separated from the insulin pump 702 including, for example, a PDM 704 of the user and/or a caregiver of the user and/or a glucose monitor 706. The communication links may include any wired or wireless communication link operating according to any known communications protocol or standard, such as Bluetooth®, Wi-Fi, a near-field communication standard, a cellular standard, or any other wireless protocol. The insulin pump 702 may also include a user interface 717, such as an integrated display device for displaying information to the user 708 and in some embodiments, receiving information from the user 708. The user interface 717 may include a touchscreen and/or one or more input devices, such as buttons, knob or a keyboard.

The insulin pump 702 includes the battery set/antenna arrangement 730 discussed above relative to FIG. 1B. The insulin pump 702 also includes the wireless transceiver 732 as mentioned above.

The insulin pump 702 may interface with a network 722. The network 722 may include a local area network (LAN), a wide area network (WAN) or a combination therein. A computing device 726 may be interfaced with the network, and the computing device may communicate with the insulin pump 702.

The drug delivery system 700 may include a glucose monitor 706 for sensing the blood glucose concentration levels of the user 708. The glucose monitor 706 may provide periodic blood glucose concentration measurements and may be a continuous glucose monitor (CGM), or another type of device or sensor that provides blood glucose or other analyte measurements. The glucose monitor 706 may be physically separate from the insulin pump 702 or may be an integrated component thereof. The glucose monitor 706 may provide the controller 710 with data indicative of measured or detected blood glucose levels of the user 708. The glucose monitor 706 may be coupled to the user 708 by, for example, adhesive or the like and may provide information or data on one or more medical conditions and/or physical attributes of the user 708. The information or data provided by the glucose monitor 706 may be used to adjust drug delivery operations of the insulin pump 702.

The drug delivery system 700 may also include the PDM 704. The PDM 704 may be a special purpose device, such as a dedicated personal diabetes manager (PDM) device. The PDM 704 may be a programmed general-purpose device, such as any portable electronic device including, for example, a dedicated controller, such as a processor, a smartphone, or a tablet. The PDM 704 may be used to program or adjust operation of the drug pump 702 and/or the glucose monitor 706. The PDM 704 may be any portable electronic device including, for example, a dedicated controller, a smartphone, or a tablet. In the depicted example, the PDM 704 may include a processor 719 and a storage 718. The processor 719 may execute processes to manage a user's blood glucose levels and for control the delivery of the drug or therapeutic agent to the user 708. The processor 719 may also be operable to execute programming code stored in the storage 718. For example, the storage may be operable to store one or more control applications 720 for execution by the processor 719. The storage 718 may store the control application 720, histories 721 like those described above for the insulin pump 702 and other data and/or programs.

The PDM 704 may include a user interface 723 for communicating with the user 708. The user interface may include a display, such as a touchscreen, for displaying information. The touchscreen may also be used to receive input. The user interface 723 may also include input elements, such as a keyboard, button, knobs or the like.

The PDM 704 may interface with a network 724, such as a LAN or WAN or combination of such networks. The PDM 704 may communicate over network 724 with one or more servers or cloud services 728. The role that the one or more servers or cloud services 728 may play in the exemplary embodiments will be described below.

As was mentioned relative to FIG. 6, the insulin pump 702 may wirelessly communicate with additional components via the battery set antenna. These additional components may include an off-body device 734. The additional components also may include a wearable device 736.

The use of the battery antenna in the system of FIG. 7 provides benefits as discussed above. Among the benefits is that it does not occupy extra surface area on the PCB, as required by conventional trace or surface mounted antennas. As a result, the PCB may be smaller than if a trace antenna or surface mounted antenna is used, and the drug delivery device may be smaller in turn. The antennas of the exemplary embodiments may be configured to be oriented substantially perpendicular to the body surface of the user so that less energy of the transmitted signals is absorbed by the human body. Antennas, like those of the exemplary embodiments, placed perpendicular to the skin surface of the user suffer less absorption by the human body that conventional trace or surface mounted antennas that are not placed perpendicular to the skin surface of the user. Some of the inefficiencies of conventional surface mounted antennas and trace antennas formed on PCBs have to do with the minimal separation of the antennas from the skin surface of the user. This may be addressed by the greater separation between the button cell batteries of the antennas of the exemplary embodiments and the skin surface of the user by, for example, design of the housing of the on-body medical device.

While the application discloses exemplary embodiments herein, it should be appreciated that various changes in form and detail may be made without departing from the intended scope as defined by the claim appended hereto.

1. A drug delivery device, comprising:
one or more button cell batteries for powering at least a portion of the drug delivery device, wherein each button cell battery of the one or more button cell batteries is cylindrical and has a longitudinal axis;
a wireless communication transceiver for transmitting and receiving wireless communications;
an electrical connection between the wireless communication transceiver and the one or more button cell batteries so that the one or more button cell batteries act as an antenna that transmit wireless communications from the wireless communication transceiver and receive wireless communications destined for the wireless communication transceiver; and
a housing that is configured to be secured to a body of a user so that the longitudinal axis of the at least one button cell battery is substantially perpendicular to a surface of the body of the user where the housing is secured.

2. The drug delivery device of claim 1, wherein the one or more button cell batteries is a single button cell battery.

3. The drug delivery device of claim 2, wherein the one or more button cell batteries are multiple button cell batteries.

4. The drug delivery device of claim 1, wherein the drug delivery device is configured to emit surface waves from the one or more button cell batteries for travelling along the surface of the body of the user.

5. The drug delivery device of claim 1, further comprising a printed circuit board on which the one or more button cell batteries are positioned and where a ground plane is formed.

6. The drug delivery device of claim 1, wherein the wireless communication transceiver is a Bluetooth transceiver, a Bluetooth Low Energy transceiver, a Body Area Network (BAN) transceiver or a WiFi transceiver.

7. The drug delivery device of claim 1 further comprising at least one battery holder for holding the one or more button cell batteries.

8. The drug delivery device of claim 7, wherein the electrical connection between the wireless communication transceiver and the one or more button cell batteries is connected to the at least one battery holder, which is in electrical contact with the one or more button cell batteries.

9. An insulin pump, comprising:
one or more button cell batteries for powering at least a portion of the insulin pump, each of the one or more button cell batteries being cylindrical and having a longitudinal axis;
a wireless communication transceiver for transmitting and receiving wireless communications;
an electrical connection between the wireless communication transceiver and the one or more button cell batteries so that the one or more button cell batteries act as an antenna that transmit wireless communications from the wireless communication transceiver and receive wireless communications destined for the wireless communication transceiver; and
a housing that is configured to be secured to a body of a user so that the longitudinal axes of the one or more button cell batteries are substantially perpendicular to a surface of the body of the user where the housing is secured.

10. The insulin pump of claim 9, wherein the one or more button cell batteries is a single button cell battery.

11. The insulin pump of claim 10, wherein the one or more button cell batteries are multiple button cell batteries.

12. The insulin pump of claim 9, wherein the insulin pump is configured to emit surface waves from the one or more button cell batteries for travelling along the surface of the body of the user.

13. The insulin pump of claim 9, further comprising a printed circuit board on which the one or more button cell batteries are positioned and where a ground plane is formed.

14. The insulin pump of claim 9, wherein the transceiver is a Bluetooth transceiver, a Bluetooth Low Energy transceiver, a Body Area Network (BAN) transceiver or a WiFi transceiver.

15. The insulin pump of claim 9, further comprising at least one battery holder for holding the one or more button cell batteries.

16. The insulin pump of claim 14, wherein the electrical connection between the wireless communication transceiver and the one or more button cell batteries is connected to the at least one battery holder, which are in electrical contact with the one or more button cell batteries.

17. A method, comprising:
positioning at least one button cell battery on a printed circuit board in a drug delivery device;
electrically connecting the at least one button cell battery to the printed circuit board to provide power for the drug delivery device;
electrically and mechanically connecting a wireless communication transceiver to the printed circuit board; and
connecting an electrical feed between the at least one button cell battery and the wireless communication transceiver to create an antenna for transmitting wireless communications from the wireless communication transceiver and receiving wireless communications for the wireless communication transceiver.

18. The method of claim 17, further comprising electrically and mechanically connecting to the printed circuit board at least one battery holder for the at least one button cell battery.

19. The method of claim 18, wherein the at least one button cell battery is held by the at least one battery holder so as to be electrically connected with the at least one battery holder and wherein the electrical feed is connected to the at least one battery holder so as to be electrically connected with the at least one button cell battery.

20. The method of claim 17, wherein the wireless communications transceiver is a Bluetooth transceiver, a Bluetooth Low Energy transceiver, a Body Area Network (BAN) transceiver or a WiFi transceiver.
